(19) United States
(12) Patent Application Publication
(10) Pub. No.: US 2017/0172484 A1
(43) Pub. Date: Jun. 22, 2017

(54) COMBINATORIAL SENSING OF SWEAT BIOMARKERS USING POTENTIOMETRIC AND IMPEDANCE MEASUREMENTS

(71) Applicants: University Of Cincinnati, Cincinnati, OH (US); United States of America as Represented by the Secretary of the Air Force, Wright-Patterson Air Force Base, OH (US)

(72) Inventors: Zachary Cole Sonner, Elsmere, KY (US); Jason C. Heikenfeld, Cincinnati, OH (US); Joshua A. Hagen, Cincinnati, OH (US)

(21) Appl. No.: 15/325,335

(22) PCT Filed: Jul. 13, 2015
(86) PCT No.: PCT/US15/40113

§ 371 Date: Jan. 10, 2017

Related U.S. Application Data
(60) Provisional application No. 62/023,232, filed on Jul. 11, 2014.

(51) Int. Cl. A61B 5/00; A61B 5/1477; A61B 5/1455; A61B 10/00; A61B 5/145; A61B 5/0205

(52) U.S. Cl. CPC A61B 5/4266; A61B 5/14517; A61B 5/14546; A61B 5/02055; A61B 5/14552; A61B 5/4875; A61B 5/6833; A61B 5/14539; A61B 5/4519; A61B 5/4866; A61B 5/7275; A61B 10/0064; A61B 5/1477; A61B 5/01

(57) ABSTRACT

A wearable sweat sensor device (1) may include a plurality of sensors (150, 160, 170, 180) capable of measuring a plurality of ion-selective biomarker potentials and a mechanism that analyzes a combination of measurements as a proxy for one or more physiological conditions such as muscle activity, exertion, or tissue damage. A device may include a sensor capable of taking at least one skin impedance measurement along with a plurality of sensors (150, 160, 170, 180) and a mechanism that analyzes a combination of measurements as a proxy for one or more physiological conditions, such as hydration or sweat rate. Because several of said sensors (150, 160, 170, 180) may not be stable when stored if fully exposed to air, the device (1) may include a temporary seal (400) for said sensors (150, 160, 170, 180) that is removable prior to placement and use of said sensors (150, 160, 170, 180).

### CROSS-REFERENCE TO RELATED APPLICATIONS

This application claims the benefit of U.S. Provisional Application No. 62/023,232, filed Jul. 11, 2014, the disclosure of which is hereby incorporated by reference herein in its entirety.

### STATEMENT REGARDING FEDERALLY SPONSORED RESEARCH OR DEVELOPMENT

The present invention was made, at least in part, with support from the U.S. Government awarded by the U.S. Air Force Research Labs and the National Science Foundation through award #1347725. The U.S. Government has certain rights in the present invention.

### BACKGROUND OF THE INVENTION

Sweat sensing technologies have enormous potential for applications ranging from athletics, to neonatology, to pharmacological monitoring, to personal digital health, to name a few applications. As noted in a recent 2014 review by Castro and colleagues titled: ‘Sweat: A sample with limited present applications and promising future in metabolomics,’ “the difficulty to produce enough sweat for analysis, sample evaporation, lack of appropriate sampling devices, need for a trained staff, and errors in the results owing to the presence of pilocarpine. In dealing with quantitative measurements, the main drawback is normalization of the sampled volume.”

There are numerous biomarkers in the body that can be used to track physiological states, including those that relate to athletics and other activities involving exertion, muscle damage, and hydration. Some of these biomarkers, such as lactate, are well-known components of sweat, however, their concentrations in sweat are not easily correlated to physiological states, since they are metabolized in the sweat gland itself (i.e., sweat levels of lactate do not reflect plasma concentrations of lactate). Similarly, Rhabdomyolysis is a syndrome characterized by muscle necrosis and the release of intracellular muscle constituents into the blood. Under Rhabdomyolysis, creatine kinase levels are typically elevated, and may partition into sweat, but creatine kinase is difficult to detect with miniaturized wearable sensors.

There are a variety of other conditions with corresponding biomarkers that emerge in sweat, but, like lactate or creatine kinase, many of these biomarkers are either not useful to measure in sweat because biomarker levels in plasma are not closely correlated to the biomarker levels in sweat or because electrical sensors to detect those biomarkers are too challenging or expensive to create. Even with the right sweat sensors, effectively determining a physiological state of the body remains a challenge for many, if not most applications.

Many of these drawbacks can be resolved by utilizing a wearable sweat sensing patch where at least the sensors are made to be intimate with the skin or to include microfluidics that are made to be intimate with the skin. Once this is enabled, numerous combinatorial measurements of relatively easy to detect sweat ions or skin parameters are possible, bringing about information and insights that would be difficult or impossible to obtain with individual measurements or multiple individual measurements. For example, one could measure five sweat or skin parameters or solutes at or near the same time, and compare those measurements in real time or how they change over time. However, this approach is not without its own challenges. For example, combinatorial measurements may require multiple sensors that must be ready to function at the same time, and therefore shelf life and use readiness of such sensors can make such measurements difficult.

### SUMMARY OF THE INVENTION

The considerable challenges described above are resolved by the present invention. The present invention provides a wearable sweat sensor device capable of measuring a plurality of ion-selective biomarker potentials with a plurality of sensors, and using a combination of said measurements as a proxy for one or more physiological conditions such as muscle activity, exertion, or tissue damage. The present invention includes embodiments with at least one skin impedance measurement along with a plurality of sensors, and using a combination of said measurements as a proxy for one or more physiological conditions, such as hydration, or sweat rate. The present invention further includes a temporary seal for said sensors which is removable prior to placement and use of said sensors, because several of said sensors may not be stable when stored ‘on the shelf’ if fully exposed to air. The sensors or patch may be stored in packaging designed to protect the item from solids, liquids or gases that may degrade the sensors during storage.

### BRIEF DESCRIPTION OF THE DRAWINGS

The objects and advantages of the present invention will be further appreciated in light of the following detailed descriptions and drawings in which:

FIG. 1 is a cross sectional view of a device according to one embodiment of the present invention positioned on skin.

FIG. 2 is a cross sectional view of a device according to one embodiment of the present invention including a sealing film to protect sensors from degradation or contamination during storage.

FIG. 3 is a cross sectional view of a device according to one embodiment of the present invention including a disposable component and a reusable component.

### DETAILED DESCRIPTION OF THE INVENTION

The invention includes reference to the International Application No. PCT/US2013/035092, the disclosure of which is included herein by reference in its entirety.

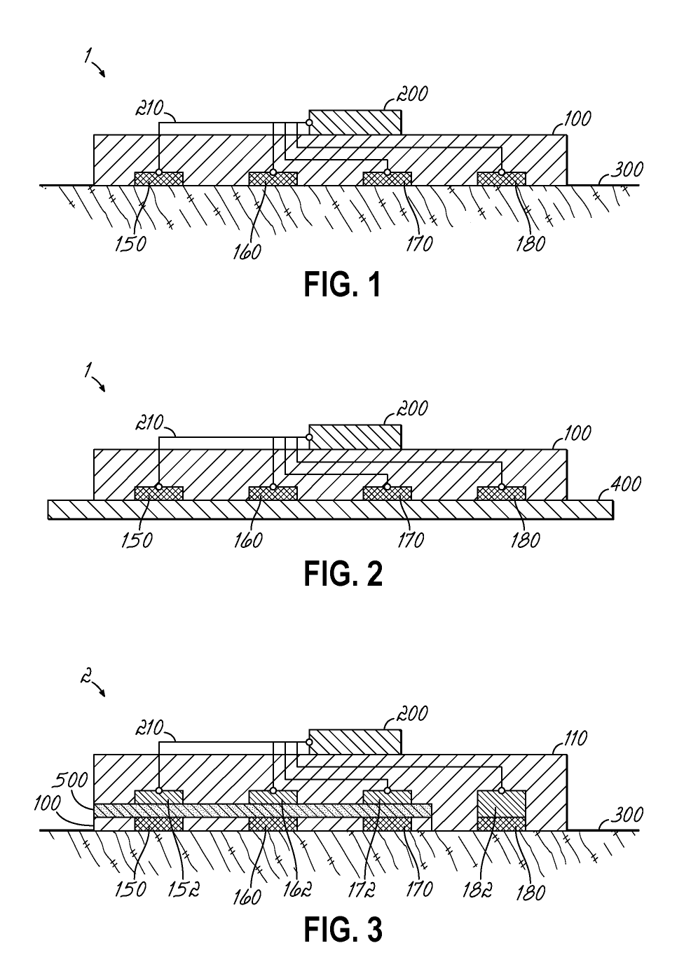

With reference to FIG. 1 a wearable sweat sensor device 1 is placed on skin 300 and includes electronics 200, a plurality of connections 210 to said electronics 200, and the connections 210 to said electronics 200 further connected to a plurality of sensors 150, 160, 170, 180. The substrate 100 supports the sensors.

With reference to FIG. 2, the device 2 is shown in a non-wearable state where it is not on skin, but is carried by the carrier element 400. Carrier element 400 may be, for example, wax paper for short-term storage or alumized mylar for longer term blockage of moisture migration. Carrier 400 can also include a pressure-sensitive adhesive to seal the carrier with sensors but also allowing the carrier to be removed. The device 1 can alternately be sealed in a container or package that provides a function similar to that of carrier 400.

The purpose of carrier 400 is to preserve function of sensors or reference electrodes that can become dehydrated, dried of solvent, or experience other degradation or contamination that could impair their performance. For example, ion-selective electrodes (ISE) can degrade if they become too dry, as the polymer can become overly crosslinked or densified, or the internal reference solution (if used) can become dry and therefore require as much as hours to become rewetted. Certain ISEs, therefore, may benefit from a seal or a backpressure of a hydrating component, such as water or other solvent vapor, during storage of the device 1 or sensors. Other solvents that may be suitable for such an application include various polar solvents, such as dimethyl sulfoxide (DMSO), or other types of non-aqueous solvents that dissolve NaCl, such as liquid ammonia.

With reference to FIG. 3, device 2 includes electronics 200, electrical connections 210, and connecting electrical pads 152, 162, 172 that are all carried by a non-disposable element 110. The non-disposable portion may also include a connecting electrical pad 182 and electrode 180, which are used to provide electrical contact or conductance with skin. The sensors 150, 160, 170 are disposable and carried by supporting material 100, which is also disposable. Adhesive, securing, or locking feature 500, such as z-axis conducting tape manufactured by 3M, is used to connect the sensors to the electrical pads. The reusable component should be configured, at a minimum, to enable and maintain good electrical contact between the wearer's skin and the device. Further, the non-disposable or reusable component is configured to couple with the sensors and/or other disposable elements during use of the device 2. Therefore, in FIG. 3, the electronics and more robust sensing electrodes, such as impedance electrodes, can be made part of a reusable component (e.g., patch, watch, bracelet, part of a shoulder pad, etc.), while the sensors can be added prior to use and disposed of afterward.

With further reference to FIGS. 1 and 2, and in an aspect of the present invention, a wearable sweat sensor with integrated sensors is made intimate with skin or microfluidics adjacent to skin, and is able to predict a variety of conditions through combinatorial potentiometric sensing of multiple solutes in sweat and by impedance measurements of skin and sweat. These types of measurements are technically achievable, especially if solutes that are generally in the millimolar range of concentrations in sweat and in the body are targeted. Although the present invention will be described in several exemplary embodiments with ion-selective electrodes and impedance measurements, those skilled in the art will recognize that other types of sensors are applicable. By way of example, potentiometric, amperometric, impedance, optical, mechanical, antibody, peptide, aptamer, or other mechanisms may be useful in embodiments of the present invention.

Embodiments of the present invention may include a computing and/or data storage mechanism capable of sufficiently analyzing the measurements taken by the sweat sensor device. The computing and/or data storage mechanism may be configured to conduct communication among system components, to monitor sweat sensor data, to perform data aggregation, and to execute algorithms capable of analyzing the sweat sensor data. By way of example, this computing mechanism may be fully or partially located on the sensing device (e.g., component 200), on a reader device, or on a connected computer network. In one embodiment, the computing mechanism may be implemented on one or more computer devices or systems. The computer system may include a processor, a memory, a mass storage memory device, an input/output (I/O) interface, and a Human Machine Interface (HMI). The computer system may also be operatively coupled to one or more external resources via the network or I/O interface. External resources may include, but are not limited to, servers, databases, mass storage devices, peripheral devices, cloud-based network services, or any other suitable computer resource that may used by the computer system.

The processor may operate under the control of an operating system that resides in the memory. The operating system may manage computer resources so that computer program code embodied as one or more computer software applications may have instructions executed by the processor. In an alternative embodiment, the processor may execute the application directly, in which case an operating system may be omitted. One or more data structures may also reside in the memory, and may be used by the processor, operating system, or application to store or manipulate data. A database may reside on the mass storage memory device and may be used to collect and organize data used by the various systems and modules described herein. The database may include data and supporting data structures that store and organize the data. The I/O interface may provide a machine interface that operatively couples the processor to other devices and systems, such as the network or an external resource. The application may thereby work cooperatively with the network or external resource by communicating via the I/O interface to provide the various features, functions, applications, processes, or modules comprising embodiments of the invention. The HMI may allow a user to interact directly with the exemplary computer.

In embodiments of the present invention, a number of sweat solutes may be targeted. A non-limiting set of targeted sweat solutes are as follows:

Sodium. In one embodiment, at least one of the sensors shown in FIG. 1 may be allocated to Na⁺. Sodium can be used to determine sweat rate (i.e., higher sweat rate results in greater detected Na⁺ amounts) as it is excreted by the sweat gland during sweating. Sodium can also be measured to mitigate its interference with other ion sensors, by using the measured Na⁺ concentration to correct errors in readings of the other ions. Additionally, Na⁺ concentration levels may be used to indicate cystic fibrosis, since Na⁺ and Cl⁻ concentrations are elevated in the sweat of such individuals.

Chloride. In one embodiment, at least one of the sensors shown in FIG. 1 may be allocated to Cl⁻. Like Na⁺, Cl⁻ can be used to determine sweat rate (i.e., higher sweat rate, greater Cl⁻ amounts) as it is excreted by the sweat gland during sweating. Chloride can also be measured to mitigate its interference with other ion sensors, by using the measured Cl⁻ concentration to correct errors in readings of the other ions. Chloride also exists at higher concentrations in the sweat of cystic fibrosis patients. Chloride can be measured using a sealed reference electrode, and therefore in some cases does not require a dedicated ion-selective electrode.

Potassium. In one embodiment, at least one of the sensors shown in FIG. 1 may be allocated to K⁺. Sweat K⁺ concentration can be used to predict K⁺ levels in blood, and in turn may indicate conditions such as dehydration, muscle activity (exertion), or tissue damage, such as Rhabdomyolysis. Low sweat K⁺ levels can indicate that an individual is at greater risk for conditions such as Rhabdomyolysis. Potassium can also be measured to mitigate its interference with other ion sensors, by using the measured potassium concentration to correct errors in readings of the other ions. Specifically, for example, K⁺ can interfere with NH₄⁺ measurements, so an accurate NH₄⁺ measurement should account for K⁺ concentration. Further, K⁺ levels in sweat are less dependent on sweat rate than are Na⁺ and Cl⁻, and therefore can improve sweat rate measurements based on Na⁺ and Cl⁻.

Ammonium In one embodiment, at least one of the sensors shown in FIG. 1 may be allocated to NH₄⁺. Ammonium can be used to predict NH₄⁺ levels in blood, and in turn may indicate conditions such as anaerobic activity level, exertion level, and may serve as a proxy indicator for serum lactate concentration. Ammonium can also be measured to mitigate its interference with other ion sensors, such as sweat pH. Further, NH₄⁺levels in sweat are less dependent on sweat rate than are Na⁺ and Cl⁻, and therefore can improve sweat rate measurements based on Na⁺ and Cl⁻. As mentioned above, K⁺ readings may interfere with NH₄⁺ sweat readings, and pH affects the partitioning of NH₄⁺ into sweat. Therefore, measuring K⁺, pH and/or sweat rate will improve the accuracy of sweat NH₄⁺ measurements.

pH. In one embodiment, at least one of the sensors shown in FIG. 1 may be allocated to measuring H⁺ activity, or pH. Sweat pH can be used to indicate sweat rate, skin health, and a variety of other conditions. Sweat pH can also interfere with other ion measurements, and therefore measuring pH is important to improve measurements of other ions.

Other ions present in sweat at millimolar-scale concentrations may also be used, including, without limitation, Ca⁺(0.28 mM), Zn⁺(4.46 mM), Cu⁺(6.3 mM), Mg⁺(34.49 mM), Fe⁺, Cr⁺, and Pb⁺. Other analytes, such as PO₄³⁻ and urea (CO(NH₂)₂), can become elevated in sweat for conditions such as renal failure and can be present at concentrations measurable by ion-selective electrodes (or an enzymatic electrode in the case of urea). Medical knowledge on the effects or interpretation of all such analyte concentrations in plasma can be similarly valued in sweat, and detected with a sweat sensor. Additional analytes of interest and their relationships are detailed in the following references: Boron, Walter F., and Emile L. Boulpaep. “Sweating.” Medical Physiology: A Cellular and Molecular Approach. 2d ed. Philadelphia, Pa.: Saunders/Elsevier, 2009.; Freedberg, Irwin M., and Thomas B. Fitzpatrick. “Biology and Function of Epidermis & Appendages.” Fitzpatrick's Dermatology in General Medicine. 5th ed. New York: McGraw-Hill, Health Professions Division, 1999. 155-63.; Goldsmith, Lowell A. “Eccrine Sweat Glands.” Physiology, Biochemistry, and Molecular Biology of the Skin. 2d ed. Vol. 1. New York: Oxford UP, 1991. 741-56.; Hurley, Harry J. “The Eccrine Sweat Glands: Structure and Function.” The Biology of the Skin. Ruth K. Freinkel. New York: Parthenon Pub. Group, 2001. 47-73.

In addition to sweat solutes, the present invention may also measure a number of other sweat parameters that used in combination with other readings improve the sweat sensor's ability to provide meaningful physiological information. These include the following non-limiting examples:

Temperature. In one embodiment, at least one of the sensors shown in FIG. 1 may be allocated to measure sensor environment temperature, skin temperature or body temperature. Temperature readings of the sensor environment, which includes the area under, or in proximity to, the sweat sensor have a significant effect on ISE function, and therefore ought to be measured and used to improve sensor measurements of solutes. In addition, skin temperature may also be indicative of various physiological states, and may be used in combination with other readings to indicate physiological states. For example, cold, clammy skin may indicate shock, dehydration, cardiac distress, and other conditions, while warm, flushed skin may indicate inflammation, stress or physical exertion. Body temperature is also an informative measure that varies according to time of day, circadian sleep cycle, fatigue, hunger, and ambient temperature. Additionally, physiological conditions such as fever, ovulation cycle, hypo/hyperthermia may be informed by body temperature, including the basal body temperature.

Sweat onset temperature. In one embodiment, at least one of the sensors shown in FIG. 1 may be allocated to measuring the sweat onset temperature. In particular, emotional sweating is triggered by neurological reactions to stress rather than reaction to high skin or body temperature. Therefore, sweat onset at low skin or body temperature may help distinguish stress sweating from other types of sweating. For example, if an individual typically starts to sweat at a skin temperature of 99.0° F., and temperature measurements indicate a skin temperature of 98.0° F., high sweat rates may indicate that stress sweating is occurring.

Impedance. In one embodiment, at least one of the sensors shown in FIG. 1 may be allocated to measuring electrical impedance of the body or skin. The spacing of the electrodes can be used to alter the depth of the impedance measurement, and to help correct for errors that result when only one pair of electrodes is used to measure impedance. For instance, closely spaced electrodes would measure impedance near the skin surface, and possibly capture an impedance measure of excreted sweat just above the skin. Electrodes placed farther apart, for example greater than 1 cm apart, would measure deeper impedances, such as body impedance. A sweat sensor patch could be placed over an area of the body, tissue, or organ, which is mainly fluid (e.g. not bone) to get an impedance measurement of the underlying bodily fluid or tissue, and thereby measure bodily hydration status. Comparing such skin surface impedance measurements to body impedance measurements may enable the sweat sensor to correct for errors in either reading, or to compare surface hydration levels to body hydration levels, among other things. In addition, impedance can be used to indicate sweat rate. Because increased sweat rates typically result in increased ion excretion, impedance levels would be expected to drop in relation to higher sweat rates.

Additionally, impedance can be used to measure several physical characteristics, sometimes requiring several frequencies of measurement, for example 5 kHz, 50 kHz & 250 kHz, and sometimes requiring that body weight be entered numerically into a readout device, such as a smartphone, that reads data from the sensor device. These characteristics may include one or more of the following: Weight & Desirable Range, Fat % & Desirable Range, Fat Mass & Desirable Range, Muscle Mass & Desirable Range, Bone Mass, BMI & Desirable Range, Physique Rating, Total Body Water %, Total Body Water Mass, Extra Cellular Water (ECW), Intra Cellular Water (ICW), ECW/ICW Ratio, BMR (Basal Metabolic Rate) & Analysis, Visceral Fat Rating, Segmental Analysis, Muscle Mass & Analysis, Fat % & Analysis, Muscle Mass Balance, Resistance/Reactance/Phase Angle.

The foregoing example uses are for stand-alone impedance measurements, however, this invention is primarily concerned with the use of impedance measurements in combination with measurement of other solutes or ions in sweat to better predict physiological condition or solute concentrations.

A device according to embodiments of the present invention may also include common electronic measurements to enhance sweat or impedance readings, such as pulse, pulse-oxygenation, respiration, heart rate variability, activity level, and 3-axis accelerometry, or other common readings published by Fitbit, Nike Fuel, Zephyr Technology, and others in the current wearables space.

The following examples are provided to help illustrate the present invention, and are not comprehensive or limiting in any manner

### Example 1

Na⁺ is measured as a proxy condition for sweat rate because Na⁺ concentration increases with sweat rate due to decreased time for Na⁺ reabsorption in the sweat duct. However, to determine if there is reference electrode drift over time, K⁺ is also measured with a second sensor. Both K⁺ and Na⁺ would share the same reference electrode. Because the concentration of K⁺ in sweat does not appreciably change with variance in sweat rate, then any drift in the reference electrode is indirectly measured. The sensor reading for Na⁺ can then be corrected for reference electrode drift.

### Example 2

K⁺ is measured as a proxy for prolonged muscle activity. K⁺ is released into the bloodstream with prolonged muscle activity or, or in the event muscle or tissue damage occurs. Since K⁺ concentration is normally relatively constant in sweat, an informative measurement of its changing concentration should be resolved according to time or sampling interval. Accordingly, a Na⁺ and/or a Cl⁻ sensor are added to the device to measure sweat rate. Sweat rate can then be used to determine the time or sampling interval for the measured K⁺ signal. As a result, a proxy for muscle activity is measured. Additionally, the time or sampling interval may also be used to determine how recently the muscle activity or damage occurred.

### Example 3

To improve measurement of NH₄⁺ concentration as a proxy for blood lactate, both K⁺ and NH₄⁺ ion-selective electrode sensors are used. NH₄⁺ is produced as part of the anaerobic cycle, and increases in the body as lactate increases. However, NH₄⁺ sensors experience significant cross-interference from K⁺, and likewise NH₄⁺ interferes with K⁺ sensors. Therefore, by comparing sensor readings for NH₄⁺ and K⁺, the sweat sensor device can account for the effects of cross-interference, and thereby improve the proxy lactate measurement.

### Example 4

With further reference to Example 3, a pH ion-selective electrode sensor is added to the device. The pH sensor improves the proxy blood lactate measurement because the sweat ratio of NH₄⁺ to NH₃ is dependent on pH. Therefore, correcting sweat NH₄⁺ for pH will provide a more accurate estimate of blood NH₄⁺ levels, thereby improving the proxy lactate measure. Further, sweat pH can become more acidic as the sweat emerges from the body and is exposed to air and carbon dioxide. Therefore, the pH ion-selective electrode may indicate how long sweat has been on the skin. Sweat rate also may affect pH, so a pH measurement may be used to estimate sweat rate. Further, pH can affect any ion reading in sweat, so a pH sensor would allow for other corrections to analyte measurements.

### Example 5

The above examples may be improved by additionally measuring skin impedance to further measure sweat rate and further improve one or more of the above measurements. For example, sweat rate can cause dilution of biomarkers that passively diffuse into sweat, or in some cases, can increase concentration of biomarkers that are actively generated by the cells in the sweat gland (e.g. Na⁺ or lactate). Sweat rate can also affect pH, and therefore an impedance sweat measurement may inform sweat pH readings.

### Example 6

With further reference to Example 3, lactate is also measured directly as a proxy for anaerobic activity in the body. However, because lactate is actively generated in the sweat gland, accurate bloodstream lactate levels must be estimated by correcting for, or minimizing, this sweat gland generated lactate. At very low sweat rates, the sweat gland lactate generation rate can be so low that sweat lactate concentration is dominated by passive diffusion of lactate into sweat from blood, thus representing a more accurate measurement of blood lactate. Similarly, higher sweat rates correspond to a higher component of gland-generated lactate compared to blood lactate. Accordingly, Na⁺ and K⁺ may be measured as a proxy for sweat rate, which would allow the device to adjust lactate readings for sweat rate.

### Example 7

A device wherein at least one sensor is capable of measuring Na⁺ concentration, at least one sensor is capable of measuring Cl⁻ concentration, at least said sensor is capable of measuring K⁺ concentration, and at least one proxy is said ion concentrations for the condition of hydration. Cl⁻ can be used to act as a stable reference electrode. Na⁺ and Cl⁻ can be used to measure sweat rate, which can be used to track water loss that could lead to dehydration. As described above, K⁺ can be used as a stable reference against Na⁺ and Cl⁻, because K⁺ does not appreciably change with sweat rate. In addition, a pH ion selective electrode can be used because sweat pH is known to change in cases of severe dehydration due to metabolic alkalosis.

### Example 8

A device with two or more ion-selective electrodes is used to measure ions in sweat as a proxy for metabolic alkalosis, with two more sensors, for example, being chosen from pH, K⁺, Na⁺, or Cl⁻, as taught in previous examples. Metabolic alkalosis is a metabolic condition in which the pH of tissue is elevated beyond the normal range (e.g., 7.35-7.45). This is the result of decreased hydrogen ion concentration, leading to increased bicarbonate, or alternatively a direct result of increased bicarbonate concentrations. Loss of hydrogen ions most often occurs via two mechanisms, either vomiting or via the kidney. Vomiting results in the loss of hydrochloric acid (hydrogen and chloride ions) along with the stomach contents. In the hospital setting, this can commonly occur from nasogastric suction tubes. Severe vomiting also causes loss of potassium (hypokalaemia) and sodium (hyponatremia). The kidneys compensate for these losses by retaining sodium in the collecting ducts at the expense of hydrogen ions (sparing sodium/potassium pumps to prevent further loss of potassium), leading to metabolic alkalosis. Hypoventilation (decreased respiratory rate) causes hypercapnia (increased levels of CO₂), which results in respiratory acidosis. Renal compensation with excess bicarbonate occurs to lessen the effect of the acidosis. Once carbon dioxide levels return to baseline, the higher bicarbonate levels reveal themselves putting the patient into metabolic alkalosis.

### Example 9

A method of determining skin impedance comprising: taking at least one measurement of skin impedance; taking at least one measurement of body impedance; and comparing said skin impedance measurement to said body impedance measurement. For example, body impedance can be measured between two electrodes placed 5 cm apart, where the electrical field path goes deep into the body. The skin impedance electrodes would be only 1 cm apart, having less depth for the electric field penetration into the body. As a result, the impedance from the further spaced electrodes can be removed via software algorithm or electronics from the impedance measured by the closely spaced electrodes, such that the main signal that is reported is skin impedance and not body impedance.

This has been a description of the present invention along with a preferred method of practicing the present invention, however the invention itself should only be defined by the appended claims.

What is claimed is:

1. A method of determining skin impedance comprising:
taking at least one measurement of skin impedance;
taking at least one measurement of body impedance; and
comparing said skin impedance measurement to said body impedance measurement.

2. The method of claim 1 further comprising:
adjusting one of said skin impedance measurement and said body impedance measurement based on the comparison of said skin impedance measurement to said body impedance measurement.

3. The method of claim 1 wherein taking at least one measurement of skin impedance includes using a sweat sensor device and taking at least one measurement of body impedance includes using the sweat sensor device.

4. The method of claim 3 further comprising:
taking at least one measurement of skin impedance using the sweat sensor device; and
determining a sweat rate using the skin impedance measurement.

5. A wearable sweat sensor device comprising:
a plurality of sensors capable of measuring a plurality of ion-selective sensor voltages;
a mechanism configured to analyze a combination of measurements of said plurality of ion-selective sensor voltages and temperature as a proxy indication of at least one physiological condition; and
a temporary seal for said sensors that is removable prior to placement and use of said sensors.

6. The wearable sweat sensor device of claim 5 including at least one sensor capable of measuring a temperature.

7. A wearable sweat sensor device comprising:
a plurality of sensors capable of measuring a plurality of ion-selective sensor voltages;
a mechanism configured to analyze a combination of measurements of said plurality of ion-selective sensor voltages as a proxy indication of at least one physiological condition;
a disposable component for a subset of said sensors; and
a reusable component for a subset of said sensors used at least for maintaining electrical continuity with skin.

8. The wearable sweat sensor device of claim 7 including at least one sensor capable of measuring a temperature.

9. A wearable sweat sensor device comprising:
a plurality of sensors capable of measuring a plurality of ion-selective sensor voltages; and
a mechanism configured to analyze a combination of measurements of said plurality of ion-selective sensor voltages as a proxy indication of at least one physiological condition.

10. The device of claim 9 further comprising:
at least one sensor capable of measuring a temperature.

11. The device of claim 9 wherein at least one said sensor is capable of measuring Na⁺ concentration and at least one said proxy is Na⁺ concentration for the condition of sweat rate.

12. The device of claim 9 wherein at least one said sensor is capable of measuring K⁺ concentration and at least one said proxy is K⁺ concentration for the conditions of muscle activity and exertion.

13. The device of claim 9 wherein at least one said sensor is capable of measuring K⁺ concentration and at least one said proxy is K⁺ concentration for the conditions of tissue damage and Rhabdomyolysis.

14. The device of claim 9 wherein at least one said sensor is capable of measuring K⁺ concentration and at least one said proxy is K⁺ concentration for the conditions of hyperkalemia and hypokalemia.

15. The device of claim 9 wherein at least one said sensor is capable of measuring Na⁺ concentration, at least one said sensor is capable of measuring Cl⁻ concentration, at least one said sensor is capable of measuring K⁺ concentration, and at least one said proxy is said ion concentrations for the condition of dehydration.

16. The device of claim 9 wherein at least one said sensor is capable of measuring NH₄⁺ concentration and at least one said proxy is NH₄⁺ concentration for the conditions of serum lactate concentration and anaerobic activity.

17. The device of claim 9 further comprising:
a sealed reference electrode to reduce measurement signal drift over time, said reference electrode including a removable covering that keeps said reference electrode hydrated until said reference electrode is applied for use.

18. The device of claim 9 further comprising:
a removable covering that keeps at least one of said plurality of sensors at the proper hydration level by keeping said sensor wetted with a hydrating component until said sensor is applied for use.

19. The device of claim 18 wherein the hydrating component is chosen from one of the following: water, a polar solvent, dimethyl sulfoxide (DMSO), and other types of non-aqueous solvents that dissolve NaCl.

20. The device of claim 9 further comprising:
a storage device that provides a backpressure of water or other solvent to preserve functionality of said sensors

21. A wearable sweat sensor device comprising:
a plurality of sensors capable of measuring a plurality of ion-selective sensor voltages;
at least one sensor capable of measuring an impedance; and
a mechanism configured to analyze a combination of measurements of said plurality of ion-selective sensor voltages and impedance as a proxy indication of at least one physiological condition.

22. The device of claim 21 further comprising at least one sensor capable of measuring a temperature.

23. The device of claim 21 wherein at least one said sensor is capable of measuring Na⁺ concentration and at least one said proxy is Na⁺ concentration for the condition of sweat rate.

24. The device of claim 21 wherein at least one said sensor is capable of measuring K⁺ concentration and at least one said proxy is K⁺ concentration for the conditions of muscle activity and exertion.

25. The device of claim 21 wherein at least one said sensor is capable of measuring K⁺ concentration and at least one said proxy is K⁺ concentration for the conditions of tissue damage and Rhabdomyolysis.

26. The device of claim 21 wherein at least one said sensor is capable of measuring K⁺ concentration and at least one said proxy is K⁺ concentration for the conditions of hyperkalemia and hypokalemia.

27. The device of claim 21 wherein at least one said sensor is capable of measuring Na⁺ concentration, at least one said sensor is capable of measuring Cl⁻ concentration, at least one said sensor is capable of measuring K⁺ concentration, and at least one said proxy is said ion concentrations for the condition of dehydration.

28. The device of claim 21 wherein at least one said sensor is capable of measuring NH₄⁺ concentration and at least one said proxy is NH₄⁺ concentration for the conditions of serum lactate concentration and anaerobic activity.

29. The device of claim 21 further comprising:
a sealed reference electrode to reduce measurement signal drift over time, the reference electrode including a removable covering that keeps said reference electrode hydrated until said reference electrode is applied for use.

30. The device of claim 21 further comprising:
a removable covering that keeps at least one of said plurality of sensors at the proper hydration level by keeping said sensor wetted with a hydrating component until said sensor is applied for use.

31. The device of claim 30 wherein the hydrating component is chosen from one of the following: water, a polar solvent, dimethyl sulfoxide (DMSO), and other types of non-aqueous solvents that dissolve NaCl.

32. The device of claim 21 wherein at least one said proxy is skin impedance for the condition of sweat rate.

33. The device of claim 21 wherein at least one said proxy is skin impedance and at least one said proxy is body impedance for the condition of hydration.

34. The device of claim 21 wherein at least one said proxy is electrolyte balance and at least one said proxy is body impedance for the condition of a risk of muscle cramping.

35. The device of claim 21 wherein said impedance electrodes are spaced at least 1 cm apart, such that deeper sensing of skin or body impedance enables hydration monitoring.

36. The device of claim 21 wherein at least one said impedance sensor has closely spaced electrodes to enable impedance measurements on or below the surface of the skin, and at least one said impedance sensor has electrodes spaced further apart to measure impedance deeper within the skin or within the body.

37. The device of claim 21 wherein the combination of measurements analyzed by the mechanism further includes at least one of the following: skin temperature, sweat onset temperature, body temperature, heart rate, pulse, pulse oximetry, accelerometry, respiration, heart rate variability, and physical activity level.

38. The device of claim 21 wherein said at least one impedance sensor is capable of using a plurality of different measurement frequencies enabling said at least one impedance sensor to indicate a plurality of different body and skin characteristics.

39. The device of claim 38 where the frequencies are chosen from at least one of the following: 5 kHz, 50 kHz, and 250 kHz.

40. The device of claim 38 wherein the combination of said measurements analyzed by the mechanism includes a measurement of a body weight of a user wearing the device.

41. A wearable sweat sensor device comprising:
a plurality of sensors capable of measuring a plurality of ion-selective sensor voltages;
a mechanism configured to analyze a combination of measurements of said plurality of ion-selective sensor voltages as a proxy indication of at least one physiological condition; and
a storage device that provides a backpressure of water or other solvent to preserve functionality of said sensors.

42. A wearable sweat sensor device comprising:
at least one sensor capable of measuring an impedance; and
a mechanism configured to analyze a combination of said measurement(s) as a proxy indication of at least one physiological condition.

43. The device of claim 41 further comprising at least one sensor capable of measuring a temperature.

44. The device of claim 41 wherein the combination of said measurements analyzed by the mechanism further includes at least one of the following: sweat electrolyte concentration, skin temperature, sweat onset temperature, body temperature, heart rate, pulse, pulse oximetry, accelerometry, respiration, heart rate variability, and activity level.

45. The device of claim 41 wherein said at least one sensor includes impedance electrodes spaced at least 1 cm apart such that deeper sensing of skin or body impedance enables hydration monitoring.

46. The device of claim 41 wherein at least one said impedance sensor has closely spaced electrodes to enable impedance measurements on or below the surface of the skin, and at least one said impedance sensor has electrodes spaced further apart to measure impedance deeper within the skin or within the body.

47. The device of claim 41 wherein said at least one impedance sensor is capable of using a plurality of different measurement frequencies enabling said at least one impedance sensor to indicate a plurality of different body and skin characteristics.

48. The device of claim 46 wherein the frequencies are chosen from at least one of the following: 5 kHz, 50 kHz, and 250 kHz.

49. The device of claim 41 wherein the combination of said measurements analyzed by the mechanism includes a measurement of a body weight of a user wearing the device.
